(19) United States
(12) Patent Application Publication
(10) Pub. No.: US 2025/0000899 A1
(43) Pub. Date: Jan. 2, 2025

(54) Methods and Compositions For Increasing Mitochondrial Biogenesis

(71) Applicant: pH Science Holdings, Inc., Everett, WA (US)

(72) Inventor: Steve Loyd, Mukilteo, WA (US)

(21) Appl. No.: 18/712,053

(22) PCT Filed: Sep. 6, 2023
(86) PCT No.: PCT/US2023/032116

§ 371 Date: May 21, 2024

Related U.S. Application Data
(60) Provisional application No. 63/404,460, filed on Sep. 7, 2022.

(51) Int. Cl. A61K 33/10; A61K 9/16; A61K 33/08; A61K 33/14; A61P 43/00

(52) U.S. Cl. CPC A61K 33/10; A61K 9/1652; A61K 33/08; A61K 33/14; A61P 43/00

(57) ABSTRACT

Compositions and methods are provided for increasing mitochondrial biogenesis. When mixed in water, the composition with a high electrical potential is a stable, dry crystalline granule containing 20 to 75 weight percent calcium carbonate. 0.1 to 10 weight percent magnesium hydroxide, and 0.5 to 10 weight percent potassium hydroxide. The granules are a crystalline matrix configured to have slow dissolution and dissociation rates when dissolved in water, which causes the solution's electrical potential to increase gradually. The negative electrical potential voltage increase between 150 and 370 mV induces mitochondrial biogenesis. The method disclosed herein includes ingesting 200 to 4,000 mg of the composition daily.

### BACKGROUND OF THE INVENTION

### 1. Field of the Invention

This invention relates to methods and compositions that increase mitochondrial biogenesis, metabolic functions, autophagy, and slows the aging process in humans.

### 2. Description of the Related Art

Mitochondria are organelles that are the primary source of energy within a cell. They are double membrane-bound structures that exist in most eukaryotic organisms. The double membranes are phospholipid bilayers and proteins. The inner membrane and the matrix enclosed by the inner membrane produce adenosine 5-triphosphate (‘ATP’) via oxidative phosphorylation, a process involving the flow of electrons through the electron transport chain.

Mitochondria occupy up to 30% of the cell's total volume in high-energy demand tissues like cardiomyocytes. In addition to their role in energy production, mitochondria are the central hub of cellular metabolism, providing metabolites for biosynthesis and producing reactive oxygen species (“ROS”), which are secondary messengers regulating intra-cellular homeostasis and allostasis.

Biogenesis of mitochondria is a self-renewal process by which new mitochondria are generated from existing mitochondria. Dysregulation of mitochondrial biogenesis is associated with aging and certain metabolic diseases. Reductions in mitochondrial density and cell function are also associated with declining health, the onset of numerous chronic diseases, and morbidity. Furthermore, declining mitochondrial function hinders energy-dependent bodily repair mechanisms, can lead to ongoing cellular dysfunction and injury, and ultimately can result in declining organ function.

Biogenesis is a complex biological process that controls organelle self-renewal and the maintenance of mitochondrial DNA (mtDNA). Ongoing research has focused on intracellular signaling pathways and proteins that induce, promote, stimulate, or inhibit mitochondrial biogenesis.

Mitochondria are renewed through biogenesis, fusion, fission, and mitophagy. Under stable conditions, mitochondria biogenesis can be induced as an adaptive response to meet energy demand from exercise or injury. Mitochondrial biogenesis is a complex process requiring coordination of nuclear NDA and mtDNA. The nuclear transcription coactivator of mitochondria biogenesis is peroxisome proliferator-activate receptor gamma coactivator-1α (PGC-1α). PGC-1α is a direct link between external physiological stimuli and the regulation of mitochondria biogenesis. Therefore, research on mitochondrial biogenesis has focused on chemicals (i.e., pharmaceuticals) to treat disease by increasing PGC-1α. Research has also focused on exercise to understand muscle bioenergetics and the health benefits of PGC-1α levels.

Oxidative phosphorylation, the driving force behind ATP production, is an electron transport process within the mitochondria. Studies of mitochondria biogenesis have developed techniques to evaluate mitochondria health and overall function. A non-trivial process has been developed to evaluate the electric potential between the mitochondrion's inner and outer membranes. The electric potential between healthy human mitochondrion's inner and outer membrane is about −160 mV.

Maintaining the mitochondria's electric potential at or near −160 mV is required for ATP production. Lower electric potentials are associated with apoptosis or cell death.

There is a need for a composition that, when properly administered, maintains the mitochondria's electric potential near or at −160 mV, thereby increasing mitochondrial biogenesis.

### SUMMARY OF THE INVENTION

The present invention is a high electric potential composition that, when appropriately administered, maintains the electric potential within mitochondria, leading to increased mitochondrial biogenesis. The term ‘high electric potential composition’ refers to compositions that produce a liquid with an electrical potential of a negative 150 mV or more when dissolved in a suitable liquid vehicle.

The composition is a slow-dissolving, high electric-potential, ingestible, crystalline electrolyte that holds an electric potential between −150 mV and −370 mV in a crystalline matrix. When administered to the human body, the composition is slowly released in two phases for up to two hours.

It is postulated that the composition delivers high negative potential mV that appears to increase PGC-1aα synthesis, thereby improving mitochondrial biogenesis and overall cellular bioenergetics. The composition is made of granular crystals that, when dissolved in water, produce an elevated electric potential mV between −150 mV and −370 mV. The granular crystals slowly release electrons so that the electric potential is elevated for one to several hours.

In one embodiment, the composition is made of granular crystals comprising an alkaline earth metal carbonate, preferably calcium carbonate; an alkali metal hydroxide, preferably potassium hydroxide; and an alkaline earth metal hydroxide, preferably magnesium hydroxide. The composition is dissolved in ultra-pure water in the presence of infrared light.

When the alkaline earth metal carbonate is calcium carbonate, the amount of calcium carbonate is 50 to 90 percent by weight of the total composition. When the alkali earth metal hydroxide is potassium hydroxide, the amount of potassium hydroxide is from 1.5 to 7.0 percent by weight of the total composition. When the alkaline earth metal hydroxide is magnesium hydroxide, the amount of magnesium hydroxide is from 0.01 to 10.0 percent by weight of the total composition. The amount of water is from 2.0 to 10.0 percent by total composition weight.

Additionally, excipients and plant-based polyphenols may be added.

Also disclosed is a method for increasing mitochondrial biogenesis in humans by administrating daily 200 to 4000 mg or more of composition.

### DESCRIPTION OF THE DRAWINGS

FIG. 1 is a graph showing the relationship between the release of electronic potential over time and the preferred embodiment of a solution. The graph demonstrates the slow mV release characteristics of the composition over 1.7 hours.

FIGS. 2-5 are electron microscope photographs of the crystalline structure of the composition.

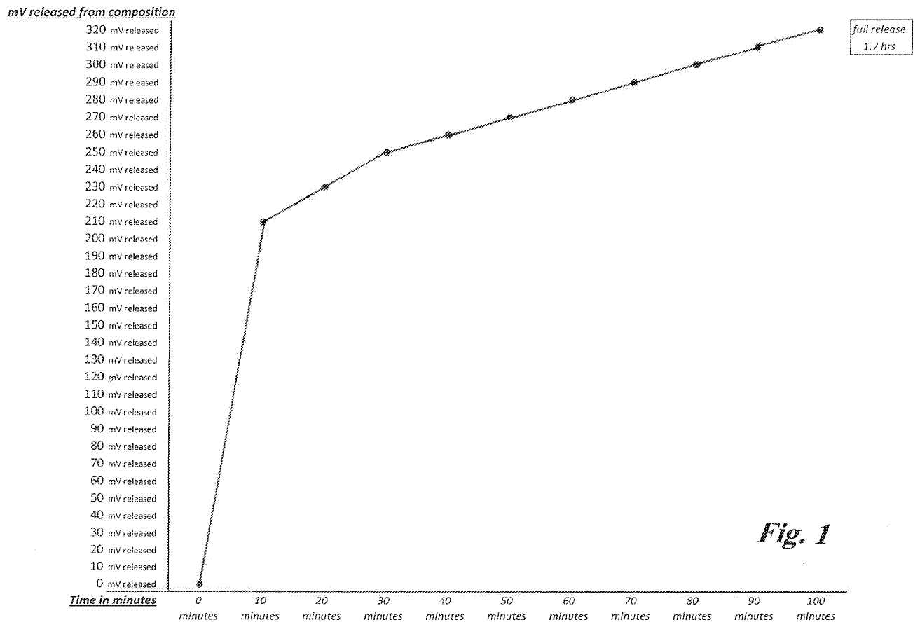

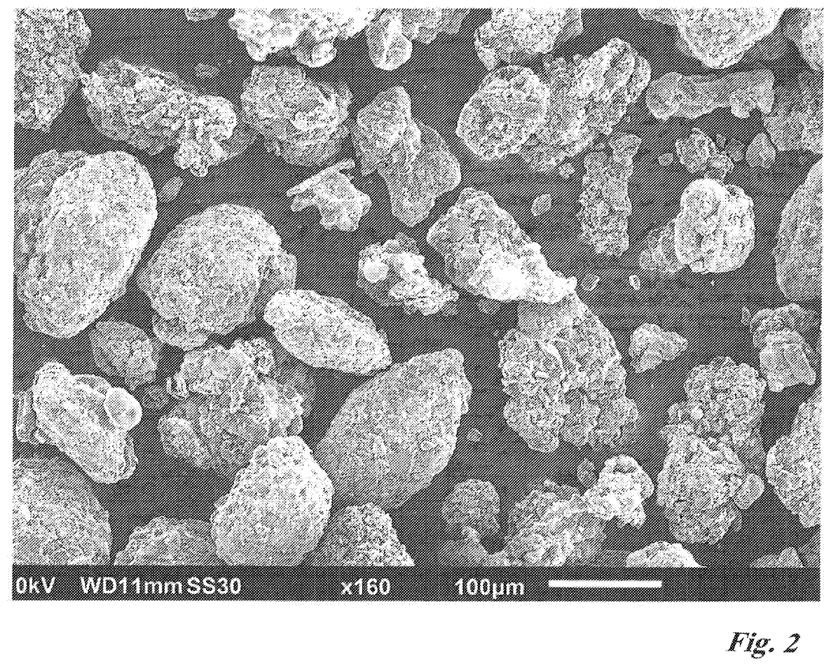

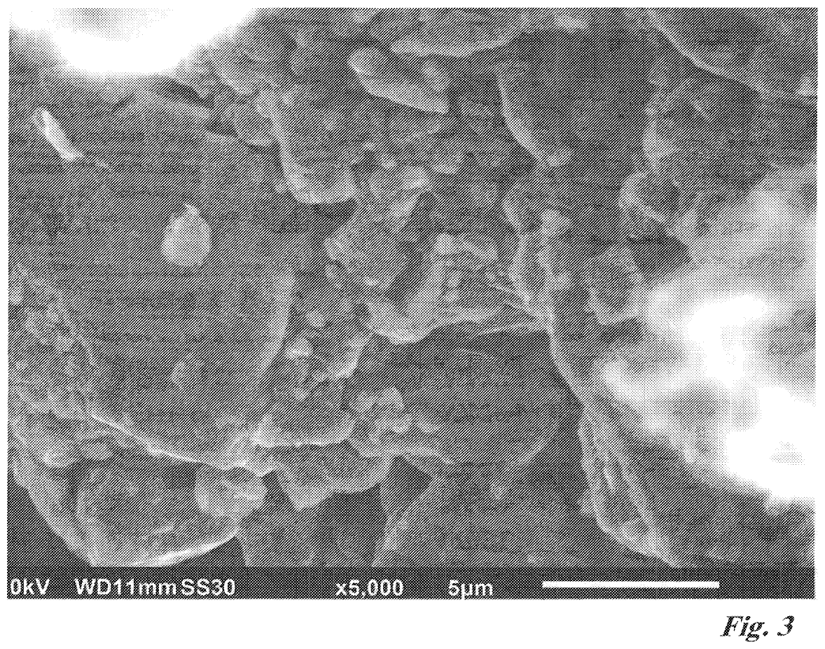

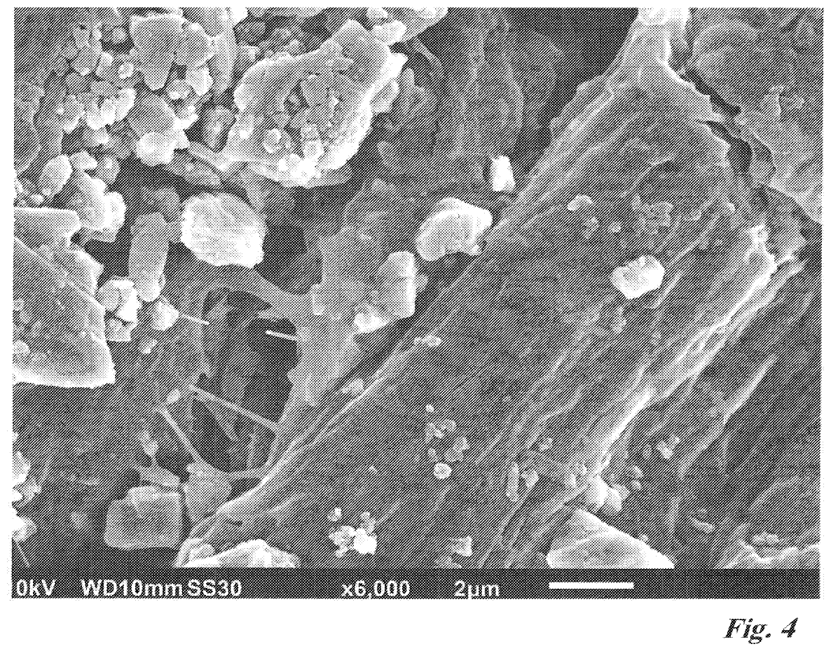

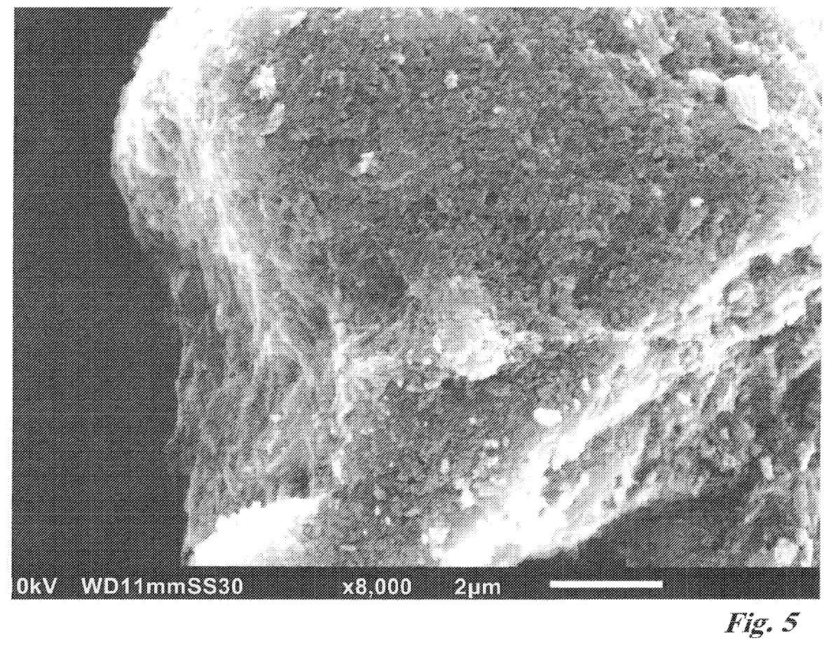

### DESCRIPTION OF THE PREFERRED EMBODIMENT(S)

The present invention generally relates to high electric potential compositions and methods for supporting mitochondrial biogenesis and attendant health and longevity benefits. As used herein, the term ‘high electric potential’ refers to electric potential existing in biological systems of a negative 150 millivolts (mV) to negative 370 mV. The terms ‘mV’ and ‘electric potential’ are used interchangeably and refer to compositions that, when added to a biological environment, change the mV significantly in that environment. In one aspect, the present invention generally relates to methods and compositions useful in changing the electric potential at the cellular level, thereby improving cellular health. The present invention is directed to methods and compositions for delivering the composition orally in a granule, tablet, capsule, emulsion, or plant-based gelatin chew.

The alkaline substances of the present invention include alkaline earth metal carbonates, alkali, and alkaline earth metal hydroxides. More specifically, alkaline earth metal carbonates include calcium and magnesium carbonates, and alkali and alkaline earth metal hydroxides include potassium and magnesium hydroxides. In one embodiment, the composition comprises calcium carbonate, potassium hydroxide, magnesium hydroxide, and potassium chloride. In suitable embodiments, calcium carbonate is present in the composition in an amount ranging from about 50% to about 70% by weight of the total composition, potassium hydroxide is present in an amount ranging from about 3% to about 8% by weight of the total composition; magnesium hydroxide is present in an amount ranging from about 0.1% to about 10% by weight of the total composition; and potassium chloride is present in an amount ranging from about 1% to about 5% by weight of the total composition.

Calcium carbonate is present in the composition in an amount ranging from about 65% to about 85% by weight of the total composition; potassium hydroxide is present in an amount ranging from about 6% to about 8% by weight of the total composition, magnesium hydroxide is present in an amount ranging from about 0.3% to about 2% by weight of the total composition, and potassium chloride is present in an amount ranging from about 2% to about 4% by weight of the total composition.

Of these electrolytes, potassium hydroxide is the most active. The remaining compounds of the preferred composition are less active. Magnesium hydroxide supplements electrolytic effects of potassium hydroxide and is the second most active electrolyte. Calcium carbonate acts as a weak electrolyte and primary component of the crystalline structure when combined with distilled water (preferably ultra-pure water) but also serves as a diluent to provide a convenient application quantity of the composition and as a calcium source. The potassium chloride is included primarily for flavor, providing a slightly salty flavor when the composition is taken orally. Combining these ingredients provides a highly effective crystalline electrolyte mixture with high electric potential, improving mitochondrial biogenesis.

The alkaline substances noted above are the active ingredients that deliver a high electric-potential ingestible composition. In addition, the alkaline substances provide additional health and nutrition benefits. For example, magnesium hydroxide in the composition has the effect of counteracting the constipation effect that often accompanies the ingestion of calcium carbonate. Furthermore, from a dietary standpoint, the alkaline substances also provide calcium, potassium, and magnesium, minerals for which the Food and Drug Administration has proposed minimum daily requirements.

In addition to the alkaline substances mentioned above, the acid-neutralizing composition of this invention may include other plant-based phytonutrients, such as flavonoids and curcuminoids. Thus, in another embodiment, the electrolyte composition includes fisetin or quercetin, ranging from about 1.0 to 10.0 percent by weight of the total composition. While polyphenols are not electrolytes, they are very difficult to absorb, and the presence of the electrolyte in the gut is likely to improve their absorption. The resulting composition can synergistically affect mitochondrial biogenesis by combining the high electric-potential, slow-release composition with a phytonutrient proven to have supportive effects at the cellular level.

The composition of this invention may also include an excipient. As used herein, the term “excipient” refers to an inert substance that forms a vehicle for the active ingredients of the composition. Suitable excipients include those that permit the effective and efficient delivery of the electrolytes and other ingredients present in the composition of this invention and include granulating and dispersing agents. For example, using a granulating agent, the composition may be formulated as a free-flowing solid, such as a powder or granules. Microcrystalline cellulose is a helpful granulating agent in rendering the composition of a free-flowing solid. Another granulating agent is fumed silicon dioxide, available from commercial sources (e.g., Cabot Corp., Tuscola, IL) and useful in controlling granule density. Furthermore, in one embodiment, the composition as a free-flowing solid is delivered to water or similar liquid and suspended until drunk.

The composition may include a dispersing agent to assist the slow dispersion of the composition into solution. A dispersing agent useful for the smooth dispersal of the composition in solution is carboxymethyl cellulose.

In general, an excipient is present in the composition, ranging from about 5.0 percent to about 35.0 percent by weight of the total composition. In one embodiment, the composition includes microcrystalline cellulose in an amount from about 1.0 percent to about 30.0 percent by weight of the total composition, carboxymethyl cellulose sodium in an amount from about 1.0 percent to about 20.0 percent by weight of the total composition.

As described above, the electrolyte composition includes alkaline substances (i.e., calcium carbonate, magnesium carbonate, potassium hydroxide, magnesium hydroxide, and water) active in delivering high electric-potential, safe, ingestible products.

Water used in the composition may be distilled, deionized, or ultra-pure water. Distilled and deionized water is easily obtained. Ultra-pure water has total organic carbon (TOC) under 10 parts per billion and 18.2 milliohms (MΩ) resistivity. Using standard lab procedures, it is produced in a class 100 clean room in an argon gas-filled hood. The compositions described below may use any of those three types of water, which will be described simply as “water.”

When water is introduced to the mixture or evaporated from the mixture to form a crystalline matrix, electromagnetic radiation (EMR) with wavelengths between 2,000 nanometers (nm) and 5,000 nm may be included in the process. The EMR is applied during the mixing process until the mixture has a spontaneous temperature rise by at least 10 degrees Celsius and preferably by 15 degrees Celsius. EMR may or may not be applied during drying to enhance crystalline stability.

Representative high negative electromotive potential granules for the present invention are described in Examples 1 and 2. Example 2 describes a representative electrolytic composition.

The composition may be formulated in a variety of ways. As stated above, the composition may be formulated as a solid, such as a powder or granule. The composition of the present invention may be granulated in the presence of the described EMR using any one of many granulation techniques known in the art. One suitable method involves mixing all the composition's dry components in distilled or high-purity water to form partially agglomerated clumps, followed by drying, chopping, and shifting to produce the desired granules. Other granulation methods well known to those of ordinary skill in the art include spray drying, extrusion, chopping, grinding, the use of a fluid bed, and high-shear granulation. The formulation of an electrolytic composition of this invention as a free-flowing granule is described in Example 1. In addition to flowing solids, the composition may also be formulated as a pill, tablet, or capsule. The composition may also be formulated as a liquid, such as an aqueous solution, slurry, emulsion, or syrup.

Used in the amounts indicated, all of the ingredients of the compositions of this invention are considered by the FDA to be generally regarded as safe (GRAS). When the composition of this invention is used as directed, including the amount of potassium hydroxide administered, it is significantly less than the upper limits established by the FDA.

The invention's compositions are designed to be slow-release, high electric-potential formulations. The high electric potential of the composition may be attributed to the presence of potassium hydroxide held in the crystalline matrix composition.

Furthermore, the compositions are held in a crystalline matrix as stable, high electro-potential granules. Furthermore, the compositions can add stable polyphenol flavonoids adjacent to but not integrated into the crystalline matrix.

The following examples further demonstrate and describe embodiments of the present invention. The examples are given solely for illustration and not limitation.

### Example 1

In this example, a representative electrolytic composition of the present invention is described. A method for combining the ingredients and formulating the composition as a free-flowing granule is also described.

| Ingredient | Percent by Weight |
|---|---|
| Calcium carbonate | 66.82 |
| Potassium hydroxide | 7.25 |
| Magnesium hydroxide | 0.67 |
| Potassium chloride | 2.67 |
| Excipient | |
| Microcrystalline cellulose | 5.33 |
| (Tabulose ™, Blenver Co., Brazil) | |
| Carboxymethyl cellulose sodium | 15.75 |
| (Solutab ™, Blenver Co., Brazil) | |
| Water | 5.51 |

### Example 2

In this example, another representative electrolytic composition of the present invention is described.

| Ingredient | Percent by Weight |
|---|---|
| Calcium carbonate | 60.33 |
| Potassium hydroxide | 10.64 |
| Magnesium hydroxide | 6.14 |
| Potassium chloride | 2.33 |
| Excipient | |
| Microcrystalline cellulose | 6.19 |
| (Tabulose ™, Blenver Co., Brazil) | |
| Carboxymethyl cellulose sodium | 8.91 |
| (Solutab ™, Blenver Co., Brazil) | |
| Water | 4.99 |

### Manufacturing Method:

A granulated formulation having the above composition was prepared as described below. To a 20-quart mixing bowl was added 3675 grams of calcium carbonate, 37 grams of magnesium hydroxide, 147 grams of potassium chloride, 293 grams of stabilizer (colloidal microcrystalline cellulose sold under the trademark TABULOSE by Blanver Farmoquimica, LTDA. Brazil) and 921 grams of cross-carmellose sodium (sold under the trademark SOLUTAB by Itacel, FarmoQuimica LTDA, Brazil). The contents of the mixing bowl were mixed dry for approximately 5 minutes. In six steps, a total of 1,700 ml of water is added during the mixing process, and mixing continues for up to two hours, accompanied by the described EMR. During one step in mixing, a solution of 399 grams of potassium hydroxide in about 400 mL of water is delivered over approximately 2 minutes to mixed solids in the mixing bowl using a peristaltic pump. After adding the potassium hydroxide solution, the blend is mixed for up to an hour or until a significant jump in the mixture's temperature. If necessary, 100 to 200 mL additional water may be added to the blend to provide a granular (i.e., non-powdery) appearance that is a moist, compressible composition. The moist formula is then mixed for approximately 20 minutes with the occasional wiping of the sides of the mixing bowl with a spatula to ensure thorough mixing of the entire formula. After thoroughly mixing, the moist formula is transferred into a large plastic bin. The bin's contents are then added in portions to fill the funnel of a cutting machine. The cutting machine and the auger are then powered on, and the moist formula is granulated, producing a granulated formula. The cutting machine and auger are then powered off, and the granulated formula was collected using a vacuum. The granulated formula is then distributed to oven trays (approximately one pound of granulated formula per tray). The trays holding the granulated formula are then placed in an oven, and the granulated formula is dried for 30 minutes at a temperature of 180° F. The trays are then rotated in the oven to ensure uniform heat treatment and dried for 30 minutes. During the mixing and drying processes listed above, the granulated formula may exposed continuously or intermittently to electromagnetic radiation (wavelengths between 2,000 nanometers (nm) and 5,000 nm).

These ingredients were combined to provide a composition that is a free-flowing granule by the method described above in Example 1. Individuals were administered 1 gm of composition per 50 lbs of body weight. Significant increases in test results were indicative of increased ATP production by the mitochondria, improved lactic acid clearance, and other indicia of improved mitochondrial function.

In compliance with the statute, the invention described herein has been described in language more or less specific as to structural features. It should be understood, however, that the invention is not limited to the specific features shown, since the means and construction shown is comprised only of the preferred embodiments for putting the invention into effect. The invention is therefore, claimed in any of its forms or modifications within the legitimate and valid scope of the amended claims, appropriately interpreted in accordance with the doctrine of equivalents.

### INDUSTRIAL APPLICABILITY

This invention has utility in industries interested in increasing mitochondrial biogenesis in living animal and plant cells, such as the medical, agricultural, and food production industries.

I claim:

1. A high electrical potential composition for increasing mitochondrial biogenesis, comprising, calcium carbonate, potassium hydroxide, and magnesium hydroxide, said calcium carbonate is present in the composition in an amount ranging from 20 to 75 percent by weight of the said composition; said potassium hydroxide is present in said composition in an amount ranging from 0.5 to 10 percent by weight of said composition; and magnesium hydroxide is present in said composition in an amount ranging from 0.1 to 10 percent by weight of said composition said composition being mixed in water to make a 2 to 10% (w/v) solution.

2. The composition of claim 1, wherein said calcium carbonate is present in the composition in an amount ranging from 25 to 45 percent by weight of said composition; said potassium hydroxide is present in an amount ranging from 1 to 5 percent by weight of said composition; and said magnesium hydroxide is present in an amount ranging from 1 to 5 percent by weight of said composition.

3. The composition of claim 1, further including potassium chloride.

4. The composition of claim 1, further including an excipient.

5. The composition of claim 4, wherein said excipient is a granulating agent.

6. The composition of claim 5, wherein said granulating agent is selected from the group consisting of: microcrystalline cellulose, croscarmellose sodium NF, and silicon dioxide.

7. The electrolytic composition of claim 1 includes a plant-based polyphenol of the flavonoid or curcuminoid family.

8. A method for increasing mitochondrial biogenesis in a human, comprising the following steps:
a. preparing a composition comprising calcium carbonate in an amount range from 20 to 75 percent by weight of the said composition, potassium hydroxide in an amount range from 0.5 to 10 percent by weight of the said composition, and magnesium hydroxide in an amount range from 0.1 to 10 percent by weight of said composition, said composition being manufactured into dry granules configured to slowly dissolve and slowly dissociate when placed in water to form an aqueous mixtures, said aqueous mixture electrical potential increases to a range 150 and 370 mV, induces mitochondrial biogenesis,
b. mixing said composition in water; and
c. orally administrating daily 200 to 4,000 mg of said composition to said human

9. The method of claim 8, wherein said high electrical potential composition the administered electrolytic composition comprises calcium carbonate in an amount range from 25 to 45 percent by weight of the said composition; potassium hydroxide, in an amount range from 1 to 5 percent by weight of the said composition; and magnesium hydroxide, present in an amount range from 1 to 5 percent by weight of the said composition.

10. The method of claim 8, wherein said potassium hydroxide in said composition in a range from 1 to 5 percent by weight of the said composition.

11. The method for manufacturing a composition for increasing mitochondrial biogenesis, comprising the following steps:
a. mixing calcium carbonate, potassium hydroxide, and magnesium hydroxide to produce a dry composition, said calcium carbonate ranging from 20 to 75 percent by weight of said composition, the amount of potassium hydroxide ranging from 0.5 to 10 percent by weight of said composition, and the amount of said magnesium hydroxide is ranging from 0.1 to 10 percent by weight of said composition;
b. adding and mixing a sufficient amount of potassium hydroxide into said dry composition;
c. adding a sufficient amount of water to produce a non-powdery, compressible composition;
d. placing said compressible composition into a cutting machine configured to cut said compressible composition into granules;
e. cutting said compressible composition into granules; and
f. drying said granules.

12. The method, as recited in claim 11, wherein after step b, said dry composition is exposed to electromagnetic radiation.

13. The method, as recited in claim 11, wherein after step c, said compressible composition is exposed to electromagnetic radiation.

14. The method, as recited in claim 11, wherein after step e, said compressible composition cut into granules is exposed to electromagnetic radiation.
